(19) United States
(12) Patent Application Publication
(10) Pub. No.: US 2021/0386695 A1
(43) Pub. Date: Dec. 16, 2021

(54) Use Of Disulfiram For Treating Infection Of SARS-COV-2

(71) Applicant: China Medical University, Taichung City (TW)

(72) Inventors: Chang-Hai Tsai, Taichung City (TW); Mien-chie Hung, Taichung City (TW); Yeh Chen, Taichung City (TW); Wen-Hao Yang, Yilan County (TW); Chia-shin Yang, Tuku Township (TW); Yu-Lin Hung, Taichung City (TW); Yu-Chuan Wang, New Taipei City (TW); Yi-Zhen Chou, Taichung City (TW); Mei-Hui Hou, Taibao City (TW); Chia-Ling Tsai, Taichung City (TW); Bao-Yue Huang, Tainan City (TW); Chian-Fang Hung, Taivhung City (TW); Hsiao-Fan Chen, Taichung City (TW); Wen-Chi Su, Taichung City (TW); Wei-Jan Wang, Taichung City (TW)

(73) Assignee: China Medical University, Taichung City (TW)

(21) Appl. No.: 17/349,023

(22) Filed: Jun. 16, 2021

Related U.S. Application Data
(60) Provisional application No. 63/039,603, filed on Jun. 16, 2020.

(51) Int. Cl. A61K 31/145

(52) U.S. Cl. CPC A61K 31/145

(57) ABSTRACT

The present disclosure relates to a method for inhibiting a binding of a SARS-CoV-2 spike protein of a SARS-CoV-2 to an angiotensin-converting enzyme 2 including contacting the SARS-CoV-2 with a sufficient concentration of disulfiram, a method for inhibiting an activity of a main protease (M^{pro}) of SARS-CoV-2 including contacting a SARS-CoV-2 with a sufficient concentration of disulfiram, and a method for inhibiting an activity of a papain-like protease (PL^{pro}) of SARS-CoV-2 including contacting a SARS-CoV-2 with a sufficient concentration of disulfiram. A method for inhibiting a replication or an infection of a SARS-CoV-2 in a cell includes contacting the cell with a sufficient concentration of disulfiram and a medical composition for use in a treatment of an infection of SARS-CoV-2 including a therapeutically effective amount of disulfiram are also provided.

### RELATED APPLICATIONS

This application claims priority to U.S. Provisional Application Ser. No. 63/039,603, filed Jun. 16, 2020, which is herein incorporated by reference.

### BACKGROUND

### Technical Field

The present disclosure relates to uses of disulfiram. More particularly, the present disclosure relates to uses of disulfiram for treating an infection of SARS-CoV-2.

### Description of Related Art

The severe acute respiratory syndrome coronavirus 2 (SARS-CoV-2) causing the current pandemic, coronavirus disease 2019 (COVID-19), has taken a huge toll on human lives and the global economy. It has since spread rapidly, infected more than one hundred and seventy-three million people globally, and caused more than 3.72 million deaths. Currently, there are no effective drugs for treatment of this disease.

The onset symptoms of COVID-19 include fever, cough, myalgia, headache, diarrhea and sputum production. Severe COVID-19 patients have developed dyspnea and acute respiratory distress syndrome (ARDS). In addition to the respiratory pathology, the infection of SARS-CoV-2 also caused injuries to heart, kidney and liver, and atrophy of spleen and lymph nodes that ultimately weakens the immune system.

While various kinds of vaccines have been available and large numbers of people have received. Less than 10 percent of the world population has received at least one dose of an approved vaccine now. At current global vaccination rates, it will take long time to achieve worldwide herd immunity against COVID-19. In addition, several novel variants of SARS-CoV-2 have been identified worldwide, particularly those termed variants of concern (VOC): B.1.351, B.1.1.7, P.1. and B.1.617, which were shown to cause vaccine escape and pose threats to antibody therapies.

Therefore, new improved vaccines and effective drugs for both the prevention and treatment of COVID-19 are urgently needed.

### SUMMARY

According to one aspect of the present disclosure, a method for inhibiting a binding of a SARS-CoV-2 spike protein of a SARS-CoV-2 to an angiotensin-converting enzyme 2 includes contacting the SARS-CoV-2 with a sufficient concentration of disulfiram.

According to another aspect of the present disclosure, a method for inhibiting an activity of a main protease (M^{pro}) of SARS-CoV-2 includes contacting a SARS-CoV-2 with a sufficient concentration of disulfiram.

According to further another aspect of the present disclosure, a method for inhibiting an activity of a papain-like protease (P129 of SARS-CoV-2 includes contacting a SARS-CoV-2 with a sufficient concentration of disulfiram.

According to still another aspect of the present disclosure, a method for inhibiting a replication or an infection of a SARS-CoV-2 in a cell includes contacting the cell with a sufficient concentration of disulfiram, wherein the SARS-CoV-2 is a wild type SARS-CoV-2, a B.1.1.7 variant or a 501Y-V2 variant.

According to yet another aspect of the present disclosure, a medical composition for use in a treatment of an infection of SARS-CoV-2 includes a therapeutically effective amount of disulfiram.

According to more another aspect of the present disclosure, a method for treating a subject suffering from COVID-19 includes administering to the subject in need of the medical composition of the aforementioned aspect.

### BRIEF DESCRIPTION OF THE DRAWINGS

The present disclosure can be more fully understood by reading the following detailed description of the embodiment, with reference made to the accompanying drawings as follows:

FIG. 1 shows a result of relative binding rate of SARS-CoV-2 spike protein to human ACE2 after treating with disulfiram at various concentrations.

FIG. 2 shows a result of relative activity of human TMPRSS2 after treating with disulfiram at various concentrations.

FIG. 3 shows a result of relative activity of SARS-CoV-2 M^{pro} after treating with disulfiram at various concentrations.

FIG. 4 shows a result of relative activity of SARS-CoV-2 PL^{pro} after treating with disulfiram at various concentrations.

FIG. 5 shows a result of the effects of disulfiram on the replication of SARS-CoV-2 replicon.

FIG. 6A shows a result of cytotoxicity assay of disulfiram in TMPRSS2-expressing Vero E6 cells.

FIG. 6B shows a result of the effects of disulfiram on the wild type, the B.1.1.7 variant and the 501Y-V2 variant of SARS-CoV-2 pseudo-particles infection in TMPRSS2-expressing Vero E6 cells.

FIG. 7A shows a result of the effects of disulfiram on the wild type and the B.1.1.7 variant of SARS-CoV-2 pseudo-particles infection in Vero E6 cells.

FIG. 7B shows a result of the effects of disulfiram on the wild type and the B.1.1.7 variant of SARS-CoV-2 pseudo-particles infection in furin-expressing Vero E6 cells.

### DETAILED DESCRIPTION

The present disclosure will be further exemplified by the following specific embodiments to facilitate utilizing and practicing the present disclosure completely by the people skilled in the art without over-interpreting and over-experimenting. However, these practical details are used to describe how to implement the materials and methods of the present disclosure and are not necessary.

I. Inhibitory Effect of Disulfiram on the Binding of SARS-CoV-2 Spike Protein to Human ACE2

<SARS-CoV-2 Spike Protein>

SARS-CoV-2 is a lipid membrane enveloped and positive-sense single-stranded RNA virus that shares significant homology with severe acute respiratory syndrome (SARS) causing the outbreak in 2003. SARS-CoV-2 encodes several open reading frames (ORFs), including four structural proteins: spike (S) protein, envelope (E) protein, membrane (M) protein and nucleocapsid (N) protein. In SARS-CoV-2, the glycosylated spike protein protrudes from the viral surface and is responsible for recognition of host receptor angiotensin-converting enzyme 2 (“ACE2” hereafter). The binding of SARS-CoV-2 spike proteins to human ACE2 triggers the membrane fusion process, followed by releasing viral genome into the host cell. Given the critical role of SARS-CoV-2 spike protein, it is considered to be one of the most important therapeutic targets for the treatment of COVID-19.

<Assessment of the Inhibitory Effect of Disulfiram on the Binding of SARS-CoV-2 Spike Protein to Human ACE2>

The effect of disulfiram on the interaction between SARS-CoV-2 spike protein and human ACE2 is measured by SARS-CoV-2 ELISA Kit. Briefly, horseradish peroxidase (HRP)-conjugate-ACE2 is pre-incubated with various concentrations of disulfiram (200 μM, 100 μM and 50 μM) at room temperature for 30 minutes, followed by addition to the ELISA plate pre-coated with the receptor-binding domain of SARS-CoV-2 spike protein at 37° C. for 1 hour. Then, TMB (3,3′,5,5′-Tetramethylbenzidine) substrate solution is then added to each well and incubated at 37° C. for 20 minutes to detect the HRP activity. The color development is stopped and the intensity is determined at OD₄₅₀. The chemical structure of disulfiram is shown as Formula (I).

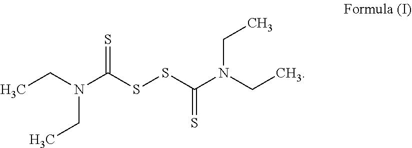 [SMILES: CCN(CC)C(=S)SSC(=S)N(CC)CC]

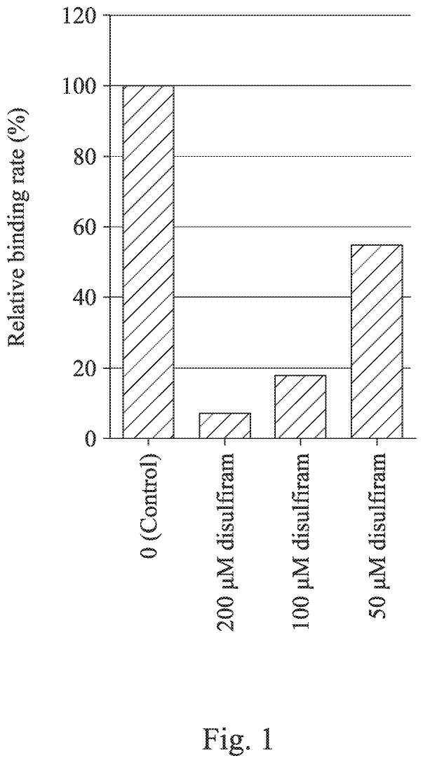

Please refer to FIG. 1, which shows a result of relative binding rate of SARS-CoV-2 spike protein to human ACE2 after treating with disulfiram at various concentrations. As shown in FIG. 1, disulfiram can significantly inhibit the interaction between SARS-CoV-2 spike protein and human ACE2. Furthermore, according to the results shown in FIG. 1, disulfiram with the concentrations of 200 μM, 100 μM and 50 μM show the inhibitory effects (100% minus the relative binding rate thereof) on binding of the receptor-binding domain of SARS-CoV-2 spike protein to human ACE2 at 92.9%, 82% and 45%, respectively. Accordingly, disulfiram has the potential to be used as a drug to prevent the infection of SARS-CoV-2 and control the spread of COVID-19.

II. Effect of Disulfiram on Protease Activity of Human TMPRSS2

<TMPRSS2 Dependent Cell Entry Pathway for SARS-CoV-2>

Once SARS-CoV-2 spike protein binds to the cell surface receptor ACE2, transmembrane serine protease 2 (“TMPRSS2” hereafter) is required for cleaving of SARS-CoV-2 spike protein at S1/S2 site and priming cell membrane fusion for viral entry. In addition to lung tissues, TMPRSS2 is also expressed in heart, kidney, liver, colon, esophagus, brain, gallbladder and testis, suggesting that the potential routes for SARS-CoV-2 infection and the manifestation of symptoms related to these organs in COVID-19 patients. Therapeutic targeting of TMPRSS2 is thought to be a good strategy to block the infection of SARS-CoV-2. Many clinically approved drugs including camostat mesylate, have been shown to be potent against SARS-CoV-2 in vitro. Therefore, the inhibitory effect of disulfiram on human TMPRSS2 has been tested.

<Assessment of the Effect of Disulfiram on the Protease Activity of Human TMPRSS2>

To access the effects of disulfiram on the serine protease activity of human TMPRSS2, the reaction mixture containing the recombinant human TMPRSS2 and various concentrations of disulfiram (200 μM, 100 μM and 50 μM) in assay buffer is pre-incubated at room temperature for 30 minutes. The fluorescent substrate is then added to start the reaction. The fluorescence signal is monitored at an emission wavelength of 440 nm with an excitation wavelength at 340 nm.

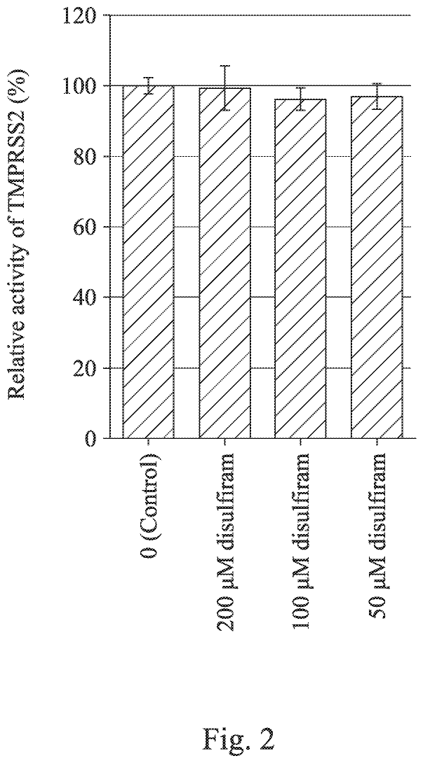

Please refer to FIG. 2, which shows a result of relative activity of human TMPRSS2 after treating with disulfiram at various concentrations. As shown in FIG. 2, disulfiram has little inhibitory on the serine protease activity of human TMPRSS2, suggesting that it is not a portent inhibitor for human TMPRSS2.

III. Inhibitory Effect of Disulfiram to SARS-CoV-2 M^{pro}

<SARS-CoV-2 Main Protease>

The ORF1 of SARS-CoV-2, made up of about two-thirds of the viral genome, could be translated into two large polyproteins, pp1a and pp1ab, that are further processed by the main protease (“M^{pro}” hereafter) and the papain-like protease (“PL^{pro}” hereafter) to generate 16 unique nonstructural proteins. SARS-CoV-2 M^{pro} is responsible for cleavage of 11 sites on pp1a and pp1ab, that are indispensable for viral replication and infection. Due to the unique substrate specificity of SARS-CoV-2 M^{pro} which do not found in human, development of effective drugs against SARS-CoV-2 M^{pro} will be a good strategy for the treatment of COVID-19 and have little or no harmful effect to human body.

<Preparation of Recombinant SARS-CoV-2 M^{pro}>

The recombinant SARS-CoV-2 M^{pro} analyzed in the present disclosure is prepared according to the following steps. First, the full-length gene encoding SARS-CoV-2 M^{pro} (ORF1ab polyprotein residues 3264-3569, GenBank code: MN908947.3) with Escherichia coli codon usage is synthesized and subcloned into pSol SUMO vector using Expresso® Solubility and Expression Screening System (Lucigen). A pET16b plasmid encoding the fluorescent protein substrate of SARS-CoV-2 M^{pro} (Hisio-mTurquoise2-TSAVLQSGFRKM-mVenus) is synthesized and constructed for fluorescence resonance energy transfer (“FRET” hereafter) based high-throughput screening assay. Each expression plasmid is transformed into E. coli BL21 (DE3) and then grown in Luria Broth medium at 37° C. until the value of OD₆₀₀ thereof reached between 0.6 and 0.8. Then, overexpression of SARS-CoV-2M^{pro} or the fluorescent protein substrate thereof is induced by adding 20% L-rhamnose or 0.5 mM IPTG and incubated for 18 hours at 20° C. After incubating for 18 hours, the cell pellets are resuspended in a sonication buffer [50 mM Tris-HCl at pH 8.0, 500 mM NaCl, 10% glycerol, 1 mM tris(2-carboxyethyl)phosphine (TCEP), 1 mM phenylmethylsulfonyl fluoride (PMSF)] and lysed by sonication on ice.

Following centrifugation at 28,000×g, 4° C. for 30 minutes, the supernatant is loaded onto a HisTrap FF column (GE Healthcare), washed by the sonication buffer containing 10 mM imidazole, and eluted with a 20 mM-200 mM imidazole gradient in the sonication buffer. An adequate amount of TEV protease is added to remove the N-terminal SUMO fusion tag of SARS-CoV-2 M^{pro}. Both TEV protease and Hiss-SUMO fusion tag are then removed by HisTrap FF column. Finally, the SARS-CoV-2 M^{pro} and the substrate protein thereof are further purified by size-exclusion chromatography and stored in buffer containing 50 mM Tris-HCl at pH 8.0, 200 mM NaCl, 5% glycerol, and 1 mM TCEP for following analysis.

<Assessment of the inhibitory effect of disulfiram to SARS-CoV-2 M^{pro}>

The inhibitory effect of disulfiram to SARS-CoV-2 M^{pro} is assessed by FRET based assay described as follows. Briefly, purified SARS-CoV-2 M^{pro} is first pre-incubated with various concentrations of disulfiram (60 μM, 30 μM, 10 μM, 5 μM and 1 μM) in the assay buffer (20 mM Tris 7.8, 20 mM NaCl) at room temperature for 30 minutes. The fluorescent protein substrate of SARS-CoV-2 M^{pro} is then added to initiate the reaction. The fluorescence signal is monitored at an emission wavelength of 474 nm with an excitation wavelength at 434 nm using Synergy™ H1 hybrid multi-mode microplate reader (BioTek Instruments, Inc.). Data are performed with two technical replicates.

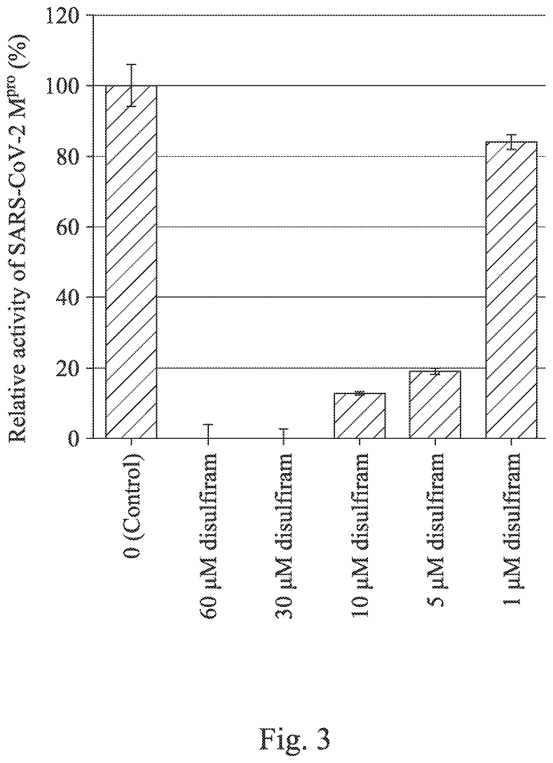

Please refer to FIG. 3, which shows a result of relative activity of SARS-CoV-2 M^{pro} after treating with disulfiram at various concentrations. As shown in FIG. 3, disulfiram shows a full inhibition on protease activity of SARS-CoV-2 M^{pro} at the concentrations of 60 μM and 30 μM. Inhibition of SARS-CoV-2 M^{pro} by 10 μM, 5 μM and 1 μM disulfiram are determined to be 87.1%, 80.9% and 16%, respectively. The half-maximal inhibitory concentration (IC₅₀) of disulfiram is estimated between 5 μM to 1 μM, indicating that disulfiram can be a highly potent drug against SARS-CoV-2 M^{pro}.

IV. Inhibitory Effect of Disulfiram to SARS-CoV-2 PL^{pro}

<Preparation of Recombinant SARS-CoV-2 PL^{pro}>

The recombinant SARS-CoV-2 PL^{pro} analyzed in the present disclosure is prepared similarly to the preparation of SARS-CoV-2 M^{pro} with some modifications and described as follows. First, the full-length gene encoding SARS-CoV-2 PL^{pro} (ORF1ab polyprotein residues 1563-1881) is optimized and synthesized with E. coli codon usage and subcloned into pSol SUMO vector using Expresso® Solubility and Expression Screening System. The expression plasmid containing SARS-CoV-2 PL^{pro} is subsequently transformed into E. coli BL21 (DE3) and grown in Luria Broth medium at 37° C. until the value of OD₆₀₀ thereof reached 0.6. Then, overexpression of target protein thereof is induced by adding 20% L-rhamnose and incubated for 16 hours to 20 hours at 16° C. Next, the bacteria are harvested by centrifugation and lysed by sonication at 4° C. The target protein is purified by immobilized metal affinity chromatography using the same procedure as described above in the section of preparation of recombinant SARS-CoV-2 M^{pro}. The TEV protease is used to remove the N-terminal SUMO tag of SARS-CoV-2 PL^{pro}. Then, the proteins are further purified by size-exclusion chromatography and stored in buffer containing 50 mM Tris-HCl pH 8.0, 200 mM NaCl, 5% glycerol, and 1 mM TCEP at −80° C. until use.

<Assessment of the Inhibitory Effect of Disulfiram to SARS-CoV-2 PL^{pro}>

To access the inhibitory effect of disulfiram on the protease activity of SARS-CoV-2 PL^{pro}, the recombinant PL^{pro} is incubated with 60 μM, 30 μM, 10 μM, 5 μM and 1 μM disulfiram at room temperature for 30 minutes. The peptide substrate (Z-RLRGG-AMC) is then added to start the reaction. The fluorescence signal is monitored continuously for 1 hour by detection of an emission wavelength 460 nm with an excitation wavelength 360 nm. Data are performed with two technical replicates.

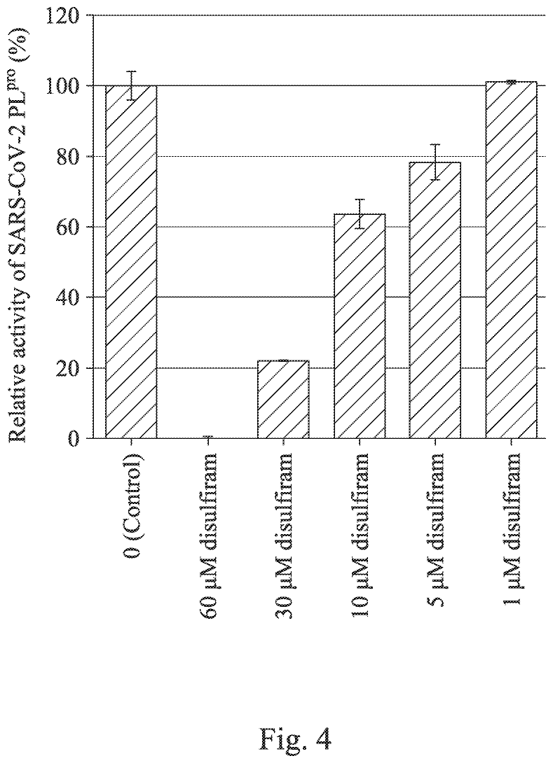

Please refer to FIG. 4, which shows a result of relative activity of SARS-CoV-2 PL^{pro} after treating with disulfiram at various concentrations. As shown in FIG. 4, disulfiram shows inhibitory effects at the concentrations of 60 μM, 30 μM, 10 μM and 5 μM on the protease activity of SARS-CoV-2 PL^{pro} at 99.9%, 77.9%, 36.3% and 21.7%. 1 μM disulfiram does not inhibit the protease activity of SARS-CoV-2 PL^{pro}. Therefore, disulfiram can be used as an effective drug against SARS-CoV-2 PL^{pro}.

V. Assessing the Inhibitory Effect of Disulfiram on the Replication or the Infection of SARS-CoV-2

In the present disclosure, it is shown that disulfiram can effectively inhibit the protease activities of both SARS-CoV-2 M^{pro} and SARS-CoV-2 PL^{pro}, that are important for viral replication of SARS-CoV-2. To detect the inhibitory effect of disulfiram on the replication or the infection of SARS-CoV-2 replication, the SARS-CoV-2 replicon is used. This replicon primarily expresses nonstructural proteins (nsp1-nsp16), neomycin resistant protein and two reporters (luciferase and green fluorescent protein). When the replicon plasmid is delivered into target cells, the luciferase reporter is expressed, and its intensity corresponds to the replication activity of SARS-CoV-2. Briefly, 0.45 μg of pBAC-SARS-CoV-2 and 0.05 μg of pCAG2.NP are co-transfected into 1×10⁵ of 293T cells. At 6 hours post-transfection, cells are re-seeded onto a 96-well plate. At 24 hours post-transfection, the cells are treated with disulfiram as indicated concentrations (2 μM and 20 μM). At 48 hours post-transfection, the luciferase activities are determined using Bright-Glo luciferase assay kit. The data are presented as percentage of DMSO control values. The values of luciferase activity represent the means±standard deviation (SD) of data from triplicate experiments.

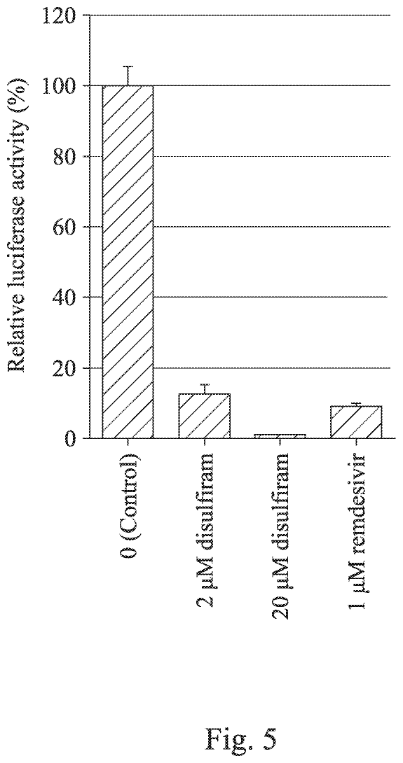

Please refer to FIG. 5, which shows a result of the effects of disulfiram on the replication of SARS-CoV-2 replicon. As shown in FIG. 5, 1 μM of remdesivir is used as a positive control because it specifically inhibits RNA-dependent RNA polymerase (RdRp) of SARS-CoV-2. As shown in the FIG. 5, disulfiram dramatically decreased the relative luciferase activity as compared to the DMSO treatment, implying that disulfiram has the potential to inhibit SARS-CoV-2 replication.

VI. Assessing the Potential Inhibitory Effect of Disulfiram on the Cell Entry by SARS-CoV-2 and its Variants

Recently, various variants of SARS-CoV-2 have been found. The B.1.1.7 variant (also known as the UK variant), which is first identified in the United Kingdom in September 2020 has D614G and N501Y mutations on spike protein and spread rapidly. The higher transmissibility of the B.1.1.7 variant is largely attributed to the N501Y mutation that increases the binding of the receptor-binding domain of SARS-CoV-2 spike protein to human ACE2. In addition, the 501Y-V2 variant (also known as the B.1.351 variant or the South African variant) is detected in South Africa in early October 2020, which contains several mutation sites, such as E484K and N501Y on spike protein. The E484K mutation is recognized as an important escape mutation that could reduce the vaccine efficiency.

To further access the inhibitory effect of disulfiram on the cell entry of SARS-CoV-2 and its variants, viral pseudo-particles (Vpp) infection assay is performed as follows. First, the TMPRSS2-expressing Vero E6 cells are generated by performing the transient transfection with the pCMV3-TMPRSS2-Flag plasmid (Sino Biology) and selecting by hygromycin. The cells are cultured in Dulbecco's Modified Eagle's Medium (DMEM) supplemented with 10% fetal bovine serum (FBS), 1×GlutaMAX, and 1% penicillin/streptomycin and incubated at 37° C. and 5% CO₂. The viability of cells after 24 hours treatment of various concentration of disulfiram is determined using the standard MTT [3-(4,5-dimethylthiazol-2-yl)-2,5-diphenyltetrazolium bromide] assay. All the treatments are done using 5×10³ cells/well in 96 wells plate. The purple formazan crystals are dissolved in DMSO (100 μL/well) and the absorbance is recorded on a microplate reader at a wavelength of 570 nm.

Viral pseudo-particles infection assay is then performed as follows. 5,000 Vero E6 cells are seeded in a 96-well plate and cultured overnight. The next day, Vero E6 cells are pre-incubated with disulfiram (1 μM, 5 μM and 10 μM) or DMSO (vehicle control) for 1 hour. Then, Vero E6 cells are infected with the viral pseudo-particles harboring SARS-CoV-2 spike protein and a luciferase reporter (purchased from National RNAi Core Facility (NRC), Academia Sinica) and followed by centrifugation at 1250×g for 30 minutes. After 24 hours incubation, the Cell Counting Kit-8 (CCK-8) assay (Dojindo Laboratories) is performed to measure the cell viability. Each sample is mixed with an equal volume of ready-to-use luciferase substrate Bright-Glo Luciferase Assay System (Promega) afterward. The relative light unit (RLU) is measured immediately by the GloMax Navigator System (Promega) and normalized with cell viability first, then the control group is set as 100% and the relative infection efficiencies are calculated.

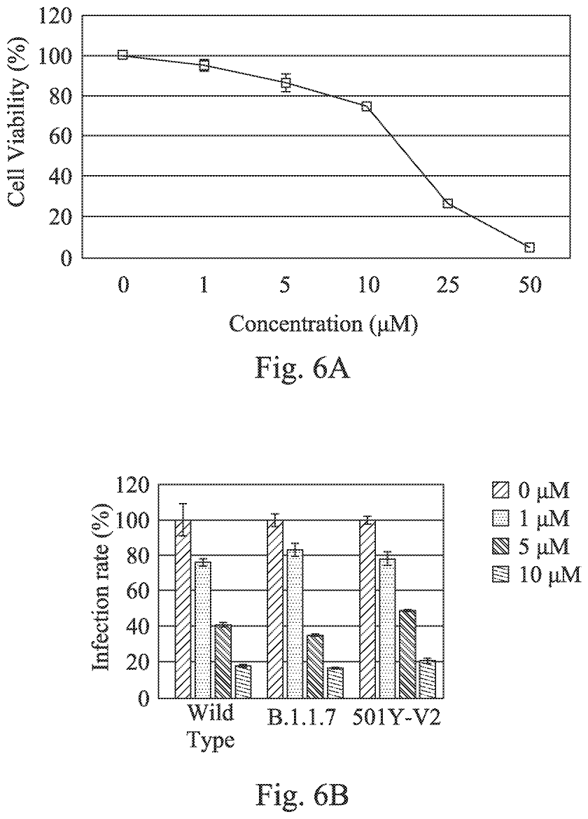

Please refer to FIG. 6A and FIG. 6B, wherein FIG. 6A shows a result of cytotoxicity assay of disulfiram in TMPRSS2-expressing Vero E6 cells, and FIG. 6B shows a result of the effects of disulfiram on the wild type, the B.1.1.7 variant and the 501Y-V2 variant of SARS-CoV-2 pseudo-particles infection in TMPRSS2-expressing Vero E6 cells. As shown in FIG. 6A, the 50% cytotoxic concentration (CC₅₀) value of disulfiram is determined to be 15.65 μM in TMPRSS2-expressing Vero E6 cells. The EC₅₀ values of disulfiram for inhibiting the wild type, the B.1.1.7 variant and the 501Y-V2 variant of SARS-CoV-2 pseudo-particles infection in TMPRSS2-expressing Vero E6 cells are further determined to be 3.10 μM, 3.18 μM and 3.84 μM, suggesting that disulfiram can efficiently inhibit the cell entry of wild type SARS-CoV-2 and the B.1.1.7 variant, but with slightly reduced inhibitory effect against the 501Y-V2 variant (FIG. 6B). Furthermore, the statistics of CC₅₀, EC₅₀ and the safety index (SI) of disulfiram against wild type SARS-CoV-2, the B.1.1.7 variant and the 501Y-V2 variant are summarized in Table 1.

TABLE 1

| Concentration of disulfiram | CC₅₀ (μM) | EC₅₀ (μM) | SI (CC₅₀/IC₅₀) |
|---|---|---|---|
| Wild type | 15.65 | 3.10 | 5.05 |
| B.1.1.7 variant | 15.65 | 3.18 | 4.92 |
| 501Y-V2 variant | 15.65 | 3.84 | 4.08 |

Moreover, in addition to the known E484K and N501Y mutations on spike protein, B.1.1.7 further contains other 6 non-synonymous mutations that modify the spike protein. One of these mutations, P681H, is located at a particular site within the spike protein at the furin cleavage site and then facilitates fusion between the virus membrane and the cell membrane. Thus, the role of the furin-dependent pathway of the SARS-CoV-2 involved in the inhibitory activity of disulfiram is further analyzed in the present disclosure.

In the present experiment, the furin-expressing cells are generated by transient transfection with the pCMV3-furin plasmid and selection by hygromycin. Vero E6 cells and furin-expressing Vero E6 cells are cultured in Dulbecco's Modified Eagle's Medium supplemented with 10% fetal bovine serum, 1× GlutaMAX, and 1% penicillin/streptomycin. Both cell lines are incubated at 37° C. and 5% CO₂. The viral pseudo-particles infection assay is then conducted as described in FIG. 6A and FIG. 6B.

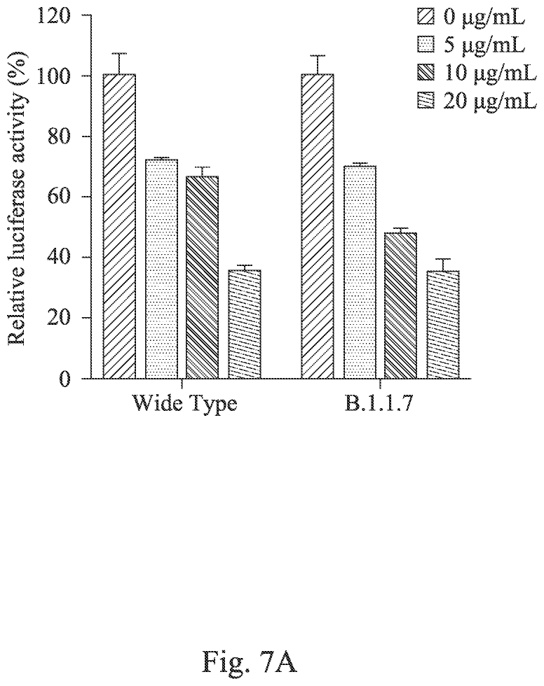

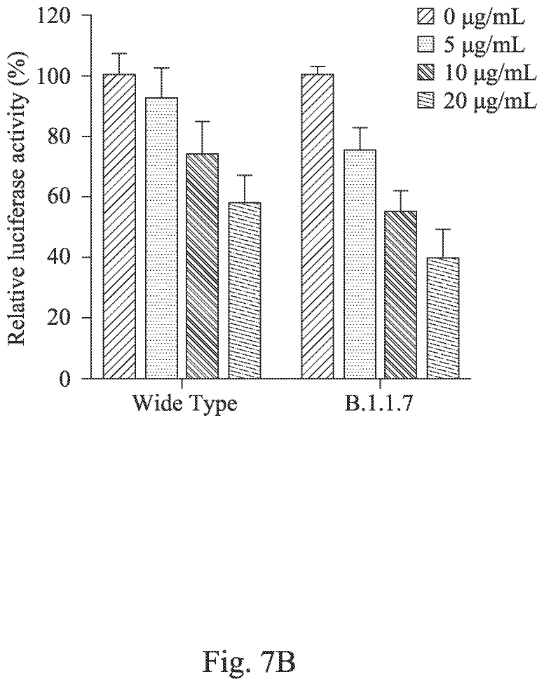

Please refer to FIG. 7A and FIG. 7B, wherein FIG. 7A shows a result of the effects of disulfiram on the wild type and the B.1.1.7 variant of SARS-CoV-2 pseudo-particles infection in Vero E6 cells, and FIG. 7B shows a result of the effects of disulfiram on the wild type and the B.1.1.7 variant of SARS-CoV-2 pseudo-particles infection in furin-expressing Vero E6 cells. As shown in FIG. 7A, disulfiram can block the wild-type SARS-CoV-2 and the B.1.1.7 variant viral pseudo-particles infection in a dose-dependent manner. However, the inhibitory capacity of disulfiram is similar by using furin-expressing Vero E6 cells compared with Vero E6 cells (FIG. 7B). Thus, it indicates that disulfiram can prevent the wild type SARS-CoV-2 and the B.1.1.7 variant viral pseudo-particles infection but not through the furin-dependent pathway.

To sum up, disulfiram can be used to manufacture a medical composition for use in prevention or treatment of an infection of SARS-CoV-2 in order to control the spread of COVID-19 or save lives from suffering this disease.

Although the present disclosure has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present disclosure without departing from the scope or spirit of the disclosure. In view of the foregoing, it is intended that the present disclosure covers modifications and variations of this disclosure provided they fall within the scope of the following claims.

What is claimed is:

1. A method for inhibiting a binding of a SARS-CoV-2 spike protein of a SARS-CoV-2 to an angiotensin-converting enzyme 2, comprising:
contacting the SARS-CoV-2 with a sufficient concentration of disulfiram.

2. The method of claim 1, wherein the sufficient concentration of disulfiram ranges from 50 μM to 200 μM.

3. A method for inhibiting an activity of a main protease (M^{pro}) of SARS-CoV-2, comprising:
contacting a SARS-CoV-2 with a sufficient concentration of disulfiram.

4. The method of claim 3, wherein the sufficient concentration of disulfiram ranges from 1 μM to 60 μM.

5. The method of claim 3, wherein the sufficient concentration of disulfiram has a half-maximum inhibitory concentration (IC₅₀ ranging from 1 μM to 5 μM.

6. A method for inhibiting an activity of a papain-like protease (PL^{pro}) of SARS-CoV-2, comprising:
contacting a SARS-CoV-2 with a sufficient concentration of disulfiram.

7. The method of claim 6, wherein the sufficient concentration of disulfiram ranges from 1 μM to 60 μM.

8. A method for inhibiting a replication or an infection of a SARS-CoV-2 in a cell, comprising:
contacting the cell with a sufficient concentration of disulfiram, wherein the SARS-CoV-2 is a wild type SARS-CoV-2, a B.1.1.7 variant or a 501Y-V2 variant.

9. The method of claim 8, wherein the sufficient concentration of disulfiram ranges from 1 μM to 20 μM.

10. The method of claim 8, wherein the SARS-CoV-2 is a pseudo-particle.

11. A medical composition for use in a treatment of an infection of SARS-CoV-2, comprising:
a therapeutically effective amount of disulfiram.

12. A method for treating a subject suffering from COVID-19, comprising:
administering to the subject in need of the medical composition of claim 11.
